(19) United States
(12) Patent Application Publication
(10) Pub. No.: US 2022/0084648 A1
(43) Pub. Date: Mar. 17, 2022

(54) CONCENTRATE DISPENSER SYSTEM

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/240,478

(22) Filed: Apr. 26, 2021

Related U.S. Application Data
(63) Continuation of application No. PCT/IB2019/058753, filed on Oct. 14, 2019.
(60) Provisional application No. 62/750,206, filed on Oct. 24, 2018.

(51) Int. Cl. G16H 20/13; G16C 20/90; A23L 33/105; A47J 31/52; B67D 7/08

(52) U.S. Cl. CPC G16H 20/13; G16C 20/90; B67D 7/08; A47J 31/521; A23L 33/105

(57) ABSTRACT

Systems and methods are provided for cannabis concentrate selection and addition to food and beverage products. The system comprises a product network server that stores information regarding a variety of cannabis-infused food and beverage products and the concentration profile for each product. The system further comprises a smart dispenser that adds specific amounts of a chosen cannabis concentrate to the food or beverage product.

### CROSS-REFERENCE TO RELATED APPLICATIONS

The present patent application is a continuation of International Application No. PCT/IB2019/058753 filed Oct. 14, 2019, which claims the priority benefit of U.S. provisional patent No. 62/750,206 filed Oct. 24, 2018, the disclosures of which are incorporated by reference herein.

### BACKGROUND OF THE INVENTION

### 1. Field of the Invention

The present disclosure is generally related to cannabis consumption consumer products. More particularly, this invention relates to how cannabis concentrates are packaged and used.

### 2. Description of the Related Art

In many jurisdictions, medical and adult recreational users are legally able to purchase cannabis products, including dried flower, extracts (e.g., oils or concentrates) or various cannabis-infused product forms, including edibles (i.e., cannabis-infused foods or beverages). In some cases, users may prefer to make their own edible products at home, often using cannabis extracts. Cannabis users may want to choose one or several cannabis concentrates and be able to precisely dose a food or beverage product with a known dosage of cannabinoids.

### SUMMARY OF THE INVENTION

The present disclosure is a system and method for at-home cannabis concentrate addition into cooking, baking and beverage mixing processes. The present disclosure includes a smart doser device attached to a liquid dispenser that adds a controlled dosage of cannabis concentrate. The smart doser device is integrated into a weighing system to determine the desired amount of cannabis concentrate. The smart doser device takes input data on the maximum temperature and duration of cooking and calculates the appropriate dosage to be added to reach the desired effects.

### BRIEF DESCRIPTIONS OF THE DRAWINGS

FIG. 1 illustrates a system for cannabis concentrate selection and addition, according to various embodiments.

FIG. 2 illustrates a concentrate database, according to various embodiments.

FIG. 3 illustrates a concentrate info module, according to various embodiments.

FIG. 4 illustrates a selection module, according to various embodiments.

FIG. 5 illustrates a dosage calculation module, according to various embodiments.

FIG. 6 illustrates a smart doser, according to various embodiments.

FIG. 7 illustrates a recipe database, according to various embodiments.

### DETAILED DESCRIPTION

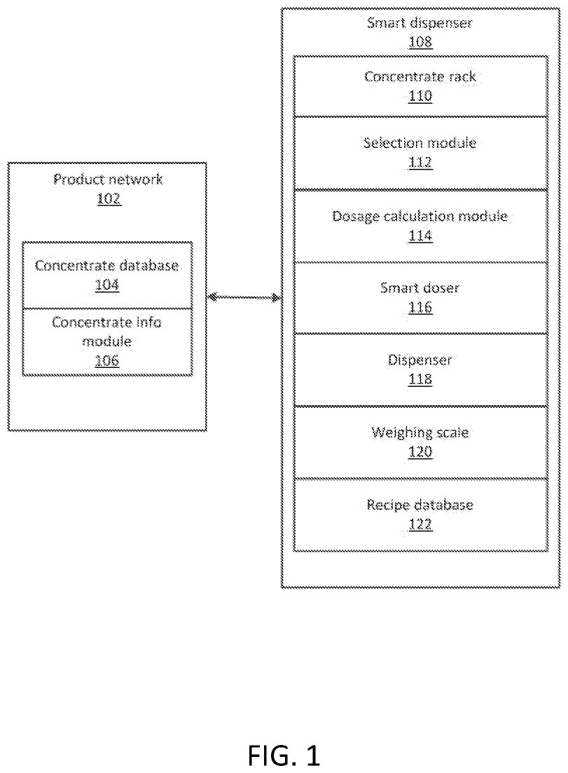

FIG. 1 illustrates a system for cannabis concentrate selection and addition, according to various embodiments. A product network server 102 stores information regarding a variety of cannabis-infused edible food or beverage products, including a concentration profile for each of the products. A concentrate database 104 is part of the product network server 102 and contains the cannabis concentrate profile data for each concentrate (e.g. potency, cannabinoid profile, terpene profile, etc.). A concentrate info module 106, part of the product network 102, is a software module that identifies the chosen cannabis concentrate from a concentrate rack 110. The concentrate info module 106 retrieves the concentration profile data for the selected concentrate(s) from the concentrate database 104 and sends the data to a dosage calculation module 114 on a smart dispenser 108.

The smart dispenser 108 automatically and dynamically adds a calculated amount of the chosen cannabis concentrate(s) to the food or beverage product. The concentrate rack 110 is part of the smart dispenser 108 and contains all the cannabis concentrates available for selection. A selection module 112 is a software module that selects the desired cannabis concentrate(s) from the concentrate rack 110 based on user input and sends the selected concentrate to the concentrate info module 106 on the product network server 102 to obtain the concentrate profile data. A dosage calculation module 114 is a software module that receives the food or beverage weight data from the weighing scale 120 and performs calculations to determine the number of additives needed for the cooking, baking or beverage-mixing process. A smart doser 116 is a smart device that adds a measured small amount of cannabis concentrate to the food or beverage product according to the amount calculated by the dosage calculation module 114. A dispenser 118 is a device that dispenses the mixed liquid into individual cups or other holding vessel. A weighing scale 120 is a device that weighs the food or beverage base product and sends the weight data to the dosage calculation module 114 to ensure accurate calculation of the amount of cannabis concentrate additive. A recipe database 122 stores various combinations of cannabis concentrates used on the smart dispenser 108 to allow future use.

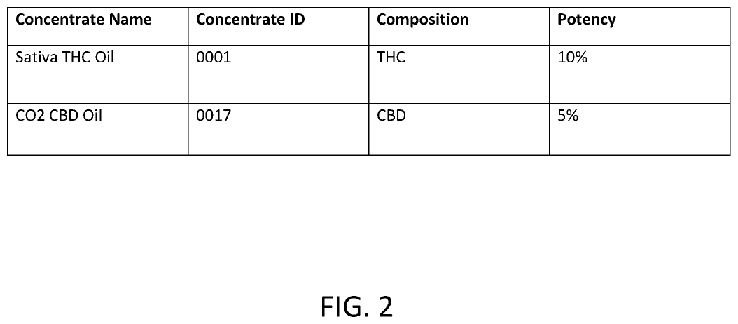

FIG. 2 illustrates a concentrate database, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The concentrate database 104 stores the concentration profile data for each cannabis concentrate. The database can include concentrate name, concentrate ID, composition and potency. For example, Sativa THC Oil, which is one type of concentrate, can be assigned a concentrate ID of 0001, be composed of tetrahydrocannabinol (THC) (a type of cannabinoid), and have a potency of 10% THC. As another example, CO2 CBD Oil, which is another type of concentrate, can be assigned a concentrate ID of 0017, be composed of cannabidiol (CBD) (a type of cannabinoid), and have a potency of 5% CBD.

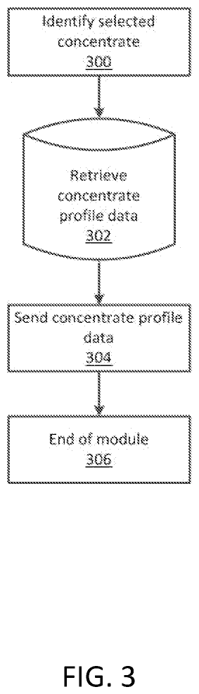

FIG. 3 illustrates a concentrate info module, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 300 where the selected cannabis concentrate(s) is identified based on user input from the selection module 112 on the smart dispenser 108. At 302, the concentrate profile data of the selected cannabis concentrate(s) is retrieved from the concentrate database 104. At 304, the concentrate profile data is sent to the dosage calculation module 114 on the smart dispenser 108. The module ends at 306.

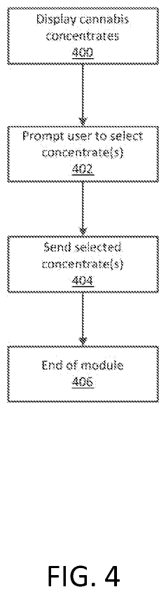

FIG. 4 illustrates a selection module, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 400 where the cannabis concentrates are displayed on the concentrate rack 110 and are available for selection. At 402, the user is prompted to select the desired cannabis concentrate(s) to be added. At 404, the selected cannabis concentrate(s) is sent to the concentrate info module 106 on the product network server 102 to allow further calculation of dosage. The module ends at 406.

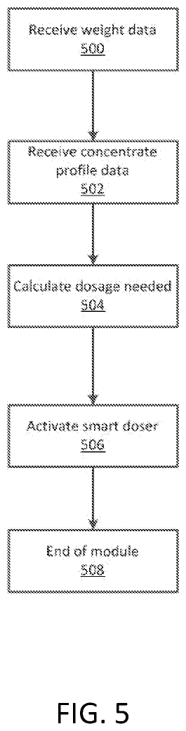

FIG. 5 illustrates a dosage calculation module, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 500 where the food or beverage weight data is received by the dosage calculation module 114 from the weighing scale 120. At 502, the concentrate profile data on quantity and potency of the selected cannabis concentrate(s) to be added is received. At 504, the dosage of cannabis concentrate needed to ensure consistent addition is calculated. At 506, the smart doser 116 is activated. The module ends at 508.

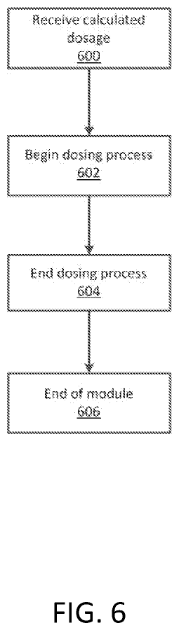

FIG. 6 illustrates a smart doser, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 600 when the smart doser 116 is activated and the calculated dosage of cannabis concentrate needed is received by the smart doser 116 from the dosage calculation module 114. At 602, the dosing process begins and the concentrate is added to the food and beverage product. At 604, the dosing process ends when the cannabis concentrate is depleted. The module ends at 606 and the smart doser 116 is deactivated.

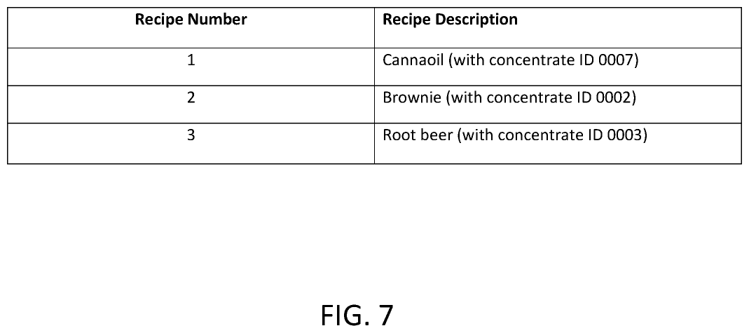

FIG. 7 illustrates a recipe database, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The recipe database 122 stores various combinations of cannabis concentrates used on the smart dispenser 108 to allow for future use. The various combinations, or recipes, are assigned a recipe number in the database for identification. For example, recipe number 2 is the combination of a brownie with concentrate ID 0002.

Although the present disclosure and its advantages have been described in detail, it should be understood that various changes, substitutions and alterations can be made herein without departing from the disclosure as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A system for cannabis concentrate selection and addition, the system comprising:
a product network server that stores information regarding each of a plurality of different concentrates; and
a smart dispenser that:
receives the information regarding at least one of the concentrates from the product network server, and
adds an amount of the at least one concentrate to at least one product based on the retrieved information.

2. The system of claim 1, wherein the product network server further includes a concentrate database in memory, wherein the concentrate database stores a profile for each of the concentrates.

3. The system of claim 1, wherein the smart dispenser further includes a concentrate rack that holds one or more available concentrates that includes the added concentrate.

4. The system of claim 3, wherein the smart dispenser further includes a selection module executable to select the at least one concentrate from the available concentrates held by the concentrate rack.

5. The system of claim 1, wherein the product network server further includes a concentrate info module executable to retrieve the information associated with the at least one concentrate.

6. The system of claim 1, wherein the smart dispenser further comprises a weighing scale that weighs the at least one product and provides a weight of the at least one product.

7. The system of claim 6, wherein the smart dispenser further comprises a dosage calculation module executable to calculate the amount of the at least one concentrate to add to the least one product based on the weight provided by the weighing scale.

8. The system of claim 1, wherein the smart dispenser further comprises a smart doser that adds the amount of the least one concentrate to the at least one product to yield a combination of product and concentrate.

9. The system of claim 8, wherein the smart dispenser further comprises a dispenser that dispenses the combination of product and concentrate into one or more vessels.

10. The system of claim 8, wherein the smart dispenser further comprises a recipe database in memory, wherein the recipe database stores a recipe of the combined product and concentrate.

11. A method for cannabis concentrate selection and addition, the method comprising:
storing information in memory regarding each of a plurality of different concentrates;
retrieving the information regarding at least one of the concentrates from the product network server, and
adding an amount of the at least one concentrate to at least one product based on the retrieved information.

12. The method of claim 11, further comprising storing a profile for each of the concentrates in a concentrate database in the memory at a product network server.

13. The method of claim 11, further comprising holding one or more available concentrates in a concentrate rack, the one or more available concentrates including the added concentrate.

14. The method of claim 13, further comprising selecting the at least one concentrate from the available concentrates held by the concentrate rack.

15. The method of claim 11, further comprising weighing the at least one product to provide a weight of the at least one product.

16. The method of claim 15, further comprising calculating the amount of the at least one concentrate to add to the least one product based on the weight provided by the weighing scale.

17. The method of claim 11, wherein adding the amount of the least one concentrate to the at least one product yields a combination of product and concentrate.

18. The method of claim 17, further comprising dispensing the combination of product and concentrate into one or more vessels.

19. The method of claim 17, further comprising storing a recipe of the combined product and concentrate in a recipe database of the smart dispenser.

20. A non-transitory, computer-readable storage medium, having embodied thereon a program executable by a processor to perform a method for cannabis concentrate selection and addition, the method comprising:
storing information in memory regarding each of a plurality of different concentrates;
retrieving the information regarding at least one of the concentrates from the product network server, and
adding an amount of the at least one concentrate to at least one product based on the retrieved information.
